(19) United States
(12) Patent Application Publication
(10) Pub. No.: US 2009/0074893 A1
(43) Pub. Date: Mar. 19, 2009

(54) Antiviral peptides

(75) Inventors: Rick de Waard, Nieuwegein (NL); Yves Pouliot, Charny (CA); Christina Juneau, Pont-Rouge (CA); Leon Franciscus Mallee, Grubbenvorst (NL)

Correspondence Address: YOUNG & THOMPSON, 209 Madison Street, Suite 500, ALEXANDRIA, VA, 22314, US

(73) Assignee: CAMPINA NEDERLAND HOLDING B.V., Zaltbommel (NL)

(21) Appl. No.: 11/919,518

(22) PCT Filed: Apr. 28, 2006
(86) PCT No.: PCT/NL2006/050103

§ 371 Date: Dec. 11, 2007

(30) Foreign Application Priority Data
Apr. 29, 2005 (EP) 05103600.2

(51) Int. Cl. A61K 36/28; A61K 38/17; A61P 31/12; A61P 31/16

(52) U.S. Cl. 424/737; 514/12

(57) ABSTRACT

The invention provides a method for the prevention or treatment of virus infections comprising administering, to a human or animal individual susceptible to or suffering from a virus infection, an effective amount of one or more casein phosphopeptides (CPP). Also provided is a composition containing such casein peptides and further agents such as vitamin C and/or nutritional components for use in treating or preventing virus infections.

The invention pertains to the use of peptides for the treatment or prevention of viral infections and to compositions to be used for such treatment or prevention.

### BACKGROUND

### Influenza

Influenza or flu is a contagious illness caused by influenza viruses. The illness is characterized as an acute infectious disease associated with respiratory and systemic symptoms, including febrile illness, characterized by tracheitis and marked myalgias, sudden onset of headache, chills, non-productive cough, sneezing, rhinorrhea, and nasal obstruction. While most healthy people recover from the flu without complications, some people, such as older people, young children, and people with certain health conditions, are at high risk for serious complications, or even death, from the flu. Epidemics of influenza typically occur during the winter months and have been responsible for an average of approximately 36,000 deaths per year in the United States during 1990-1999.

Influenza viruses are pleomorphic and belong to the family of orthomyxo-viruses. The family includes influenza virus A, B and C. The viruses are enveloped, spherical particles. Filamentous forms may also occur. The internal antigens, M1 and nucleoprotein antigens, are the type-specific proteins which are used to determine whether a particular virus is A, B or C. The external antigens, hemagglutinin and neuraminidase, show more variation and are the subtype and strain-specific antigens. The proteins are encoded by eight RNA strands.

Type A viruses are found in many kinds of animals, including birds, pigs and humans. Influenza virus A is held responsible for the influenza pandemics of 1918, 1957 and 1968. The type B virus widely circulates in humans. Type C has been found in humans, pigs, and dogs and causes mild respiratory infections, but is not known to set off epidemics. The influenza virus is spread host to host via small particle aerosols, which can get into respiratory tract.

Influenza viruses are pleomorphic because they mutate constantly due to antigenic drift and shift. Repeated minor antigenic changes in the hemagglutinin and neuraminidase, which generate strains that retain a degree of serologic relationship with the currently prevailing strain, are called antigenic drift. A major change in hemagglutinin and/or neuraminidase that yields an antigen showing no serologic relationship with the antigen of the strains prevailing at the time is called antigenic shift. Antigenic shift has been related to the above mentioned influenza pandemics.

The immune mechanisms responsible for recovery from influenza have not been clearly defined. The respiratory tract has a series of protective mechanisms against influenza infection, including the mucin layer, ciliary action, and protease inhibitors that may prevent effective cell entry and virus uncoating. Once the epithelial cell is infected, proinflammatory cytokines, notably interleukin-6 and IFN-α, are induced and released from these cells. Other cytokines appear later, such as IL-8 and TNF-α. The type 1 interferons (IFN-α and -β) are key components of antiviral host defense. Produced within hours following viral infection, IFN-α/β induces an antiviral state in uninfected cells that helps to contain spreading of the virus. IFN-α/β also activates natural killer cells (NK) and macrophages.

Current anti-viral strategies use three main directions: (i) Vaccination; (ii) Chemotherapy (i.e. antiviral drugs) and (iii) Natural products, including COLD-FX® (a proprietary extract isolated from North American Panax quinquefolium root), Zinc, Vitamin C and Echinacea.

Vaccines have a short-lived protective effect. They need to be given every year because of this short lived-nature of the protection, but also since the most effective strains for the vaccine will change due to antigenic drift or shift of the prevalent influenza strains.

Antiviral drugs are usually associated with side effects. For instance, the most common side effects of oseltamivir consist of nausea and vomiting. Side effects of rimantadine and amantadine may include headache, dizziness, insomnia, irritability, trouble concentrating, and anxiety.

WO 95/32727 describes inhibition of infection by respiratory syncytial virus (RSV) using optionally hydrolysed human β-casein or its recombinant form, in infant formulae. Human milk products are not easily accessible on an industrial scale, while recombinant products are often undesired as being non-natural.

WO 97/26320 discloses a nutritional product for inhibiting adhesion of the bacterium Haemophilus influenzae to human cells, containing recombinant phosphorylated human β-casein. However, the adhesion of bacteria is unrelated to infection by a virus and moreover, the disease caused by H. influenzae (meningitis) is unrelated to typical virus infections such as influenza. In this respect, Aniansson et al. (Microbial Pathogenesis 1990, 8, 315-323) have shown that, contrary to human analogues, casein and whey fraction from bovine milk are inactive in inhibiting adhesion of the bacteria Streptococcus pneumoniae and H. influenzae.

Thus there is a serious need for developing new antiviral agents based on natural products, which are effective against a broad spectrum of influenza and other viruses and have no or acceptable side-effects. It is especially desired to provide compositions that can be effectively used against virus infections such as influenza and cold by self-medication, i.e. without a prescription being mandatory.

### DESCRIPTION OF THE INVENTION

It was found according to the invention that certain casein-derived peptides are effective in preventing and treating viral infections. The invention concerns the use of casein peptides, in particular casein phosphopeptides (CPP) for preparing a nutritional or pharmaceutical composition for the prevention or treatment of a virus infection. The invention also concerns methods for the prevention or treatment of virus infections comprising the administration to an animal, such as humans, livestock (horses, pigs etc.), poultry, preferably a mammal, most preferably a human, susceptible to or suffering from a virus infection of an effective amount of one or more casein peptides.

### Casein Phosphopeptides

Casein peptides to be used according to the invention are peptides derived from casein, preferably non-human casein in particular casein from ungulates, especially ruminants, more in particular members from the family of the Bovidae. The Bovidae include cattle and allies (Bovinae) and goats and allies (Caprinae). Preferred Bovinae species include cattle, yak, buffalo and water buffalo; preferred Caprinae species include sheep and goat. Most preferably the casein is bovine, sheep, goat or yak casein, especially bovine casein. The casein peptides comprise an amino acid sequence of at least 2 up to about 50 amino acid residues. Particularly useful peptides contain from 3 up to about 25 amino acid residues. Particularly preferred are casein phosphopeptides (CPP), which are defined herein as casein-derived peptides having at least one phosphoserine residue per peptide molecule. It is preferred that, on average, the CPP contains at least 1 phosphoserine (SerP) residue per 20 amino acid residues, more preferably at least 1 SerP residue per 10 amino acid residues or even at least 1 SerP per 7, and e.g. up to 3 SerP per 7. In addition to or instead of SerP, other phosphorylated amino acids, such as phosphothreonine (ThrP) or phosphotyrosine (TyrP) may be present. The phosphorus content of the CPP is preferably between 0.6 and 4.8 wt %, more preferably between 2.5 and 4.5 wt %. The nitrogen to phosphorus w/w ratio is preferably between 2.2 and 20, more preferably between 2.4 and 4.3. Suitable CPP can have a phosphorus content between 0.6 and 1.5, especially between 0.7 and 1.3 wt %, with a N/P ratio between 10 and 20, especially between 13 and 17; these are sometimes referred to as CPP 1. The high-phosphorus CPP, having a phosphorus content between 2.5 and 4.5 wt %, are referred to as CPP 3 type of CPP.

Examples of preferred CPP are those comprising the bovine αS₁-casein amino acid sequence 43-58, 59-79 and 106-119, αS₂-sequences 2-21, 47-70, 126-137 and 138-149, or β-casein sequence 2-25, or parts thereof comprising at least one, preferably at least two SerP residues.

Suitable CPP can be prepared by enzymatic hydrolysis of casein or caseinate, especially whole casein, α-caseins, κ-casein or β-casein, for example using trypsin, pepsin, chymotrypsin, pancreatin or bacterial (Bacillus), fungal or plant exoproteases or mixtures thereof. Preferred degrees of hydrolysis are between 1 and 60%, more preferably between 5 and 40%, most preferably between 10% and 25%, respectively, resulting in average peptide lengths of between 100 and 3 amino acids, preferably between 40 and 5 amino acids, more preferably 25-8 amino acids. Most preferred is an average peptide length of between 12 and 4 amino acids. Without further fractionation, the peptide mixture thus produced will contain between 15 and 30% of CPP's, and such a mixture can be suitably used as such according to the invention. Unfractionated CPP is generally called CPP 1.

It is preferred, however, to use a peptide mixture enriched in CPP, so as to contain at least 50% of CPP, up to e.g. 90% or even 100%. Methods of increasing CPP content from peptide mixtures are known in the art, such as anion exchange chromatography (e.g. using cationic Sepharose®), calcium or barium precipitation, ultrafiltration/diafiltration and the like. The production and fractionation of CPP is described e.g. in WO 94/06822. Such a purified CPP is generally called CPP 3. CPP 3 type of peptides may comprise peptides or peptide mixtures with an average peptide length as specified for CPP 1 type products.

The casein peptides or casein phosphopeptides may be part of a mixture comprising other proteins, in which mixture the other proteins may be hydrolysed or intact. These other proteins may be CPP of different qualities or other phosphorus-containing or other peptides.

CPP analogues, e.g. obtained by chemical or genetic modification of casein-derived peptides, or obtained from other, preferably natural phosphopeptides such as phosphovitin or plant phosphopeptides having the required phosphorus content and chain length, can also be used instead of, or in addition to, the CPP described above. Furthermore, synthetic peptides containing SerP residues may be used.

The CPP to be used according to the invention preferably have a low calcium content, i.e. the phosphate groups should preferably be essentially free from multivalent cations. Especially, the calcium content is below 1.0 wt. %, more particularly below 0.1 wt. %, most preferably below 0.05% on a protein/peptide basis. It is preferred that the Ca/P ratio of the CPP is below 0.3, preferably below 0.1, most preferably below 0.03 (w/w).

### Other Components

The composition to be administered according to the invention may contain, in addition to the CPP, other active components, nutritional ingredients, excipients, flavours, colorants, stabilisers and other conventional components for a nutritional or pharmaceutical composition.

Other active components that may be combined with the CPP especially include natural or nature-derived products, such as vitamins, especially vitamin C, lactoferrin or transferrin, glycomacropeptide, echinacea, etc. It is preferred that the weight ratio between CPP and the other component(s) is at least 0.25:1, for example between 1:1 and 9:1.

The CPP-containing composition may be formulated in a way which is conventional for oral pharmaceutical or nutritional compositions. Where the composition to be administered is a nutritional composition, it may be as a food supplement, bar, drink, yoghurt, sweet, gum, etc. Such food products contain carbohydrates and/or proteins, optionally together with further food components, such as fats, fibers, vitamins, minerals, and optional additives such as flavours, sweeteners, stabilisers and the like. Preferably the weight ratio between CPP and the carbohydrate and/or protein is between 1:2 and 1:50, more preferably between 1:5 and 1:20.

Where the composition is a pharmaceutical composition, it may be in a form suitable for oral, nasal, or other way of administration. It may e.g. be a tablet, granule, powder, syrup, capsule, solution, gel, lozenge, inhalant spray, nasal spray etc., containing conventional excipients or carriers, such as water, starch or starch fractions, microcrystalline cellulose or cellulose derivatives, pectin, other polysaccharides, lactose, other sugars, etc. Solids dosage forms may also be coated with an enteric coating.

### Administration

The dosage level will depend on various individual factors, such as age, physical and nutritional condition and nature of the virus. Typical dosage levels will be in the range of between 100 mg and 10 g CPP per individual per day, preferably between 200 mg and 5 g, per individual per day. In terms of body weight, the dosage levels are generally between 1 and 1000 mg CPP per kg per day, preferably between 5 and 750 mg per kg per day. The daily dosages may be administered in a single dosage unit or, preferably, over multiple daily dosages, e.g. 2-4 times a day.

### Viruses

In a preventive or therapeutic fashion CPP are active against a wide range of viruses, including the Orthomyxoviruses (a family consisting of influenza virus A, B and C) as well as Rhinoviruses, Picornaviruses, Enteroviruses, Paramyxoviridae, Adenoviruses, Rotaviruses, Astroviruses, Norwalkviruses, Coronaviruses, and Noroviruses. Thus, the CPP can be used for the prevention and treatment of viral diseases such as influenza, common cold, and other infections of the respiratory tract.

### EXAMPLES

### Example I

### Preparation and Fractionation of CPP

Sodium caseinate (5% aqueous solution) was hydrolysed using trypsin for about 3 hours at pH 8.0 and 37° C. The enzyme was subsequently thermally inactivated. This caseinate hydrolysate was treated with activated carbon, filtered and dried to obtain a CPP product. The product had the following characteristics: 95.3% dry matter; 87.3% protein; 13.68% total nitrogen (N); 0.92% total phosphorus (P); N/P 14.9; CPP 21%; it is referred to as CPP 1. Comparable products are commercially available: CE 90 CPP; DMV International.

Sixteen kg of such a casein hydrolysate was loaded as a 1% aqueous solution onto a Sepharose Fast Flow column and eluted with 0.7 M NaCl solution, pH 5.5. The eluate was concentrated, delactosed and desalted using an AFC-30 polyamide membrane at a pressure of 15-18 bar. After bacteriological purification (microfiltration) the product was dried by spray-drying. The product had the following characteristics: 93.8% dry matter; 87.3% protein; 10.8% total nitrogen; 3.7% total phosphorus; N/P 2.9; calcium content 0.015%, CPP 90.5% on protein basis (83% on total product basis). The product was referred to as CPP 3.

### Example II

### In Vitro Data CPP Studies

### Virus Plaque Assay

Confluent monolayers of MDCK cells in six-wells tissue culture plate were inoculated with human influenza virus strains (A/Sydney/97 (H3N2), A/Beijing/262/95 (H1N1) and B/Harbin/07/94) diluted in serum-free MEM. Prior to inoculation, the medium was decanted and the cells were washed twice with pre-warmed phosphate-buffered saline (PBS). After adsorption at room temperature, the cell monolayers were overlaid with defined cell culture medium containing 0.8% agarose. CPP and CPP3 were tested in duplicate at concentrations of 10 and 1000 μg/ml. BSA was used as control.

Three modes of exposition were studied: MDCK cells were exposed during 1 h with CPP either before (pre-exposure) or after (post-infection) the infection, or cells and virus were exposed separately to CPP during 1 h before the infection (pre-incubation). Two wells of each plate were overlaid with medium alone so that the number of plaques formed in the absence of products could be determined. Once overlaid, the plates were incubated at 37° C. in 5% CO₂. After 36 to 45 h (depending on the used virus strains), the plates were fixed with 3.7% formaldehyde and stained with crystal violet. The plaques were counted and the percentage of plaque reduction for each dilution of products was determined. Results were expressed as the percentage of virus survival:

$$Virus survival ( \% ) = \frac{100 - [ ( \begin{matrix}P F U without molecule - \\ P F U with molecule\end{matrix} ) ]}{P F U without molecule} \times 100$$

$$( P F U = plaque forming units )$$

The same experiments with the same amounts of BSA (control), instead of CPP or CPP3, resulted in a virus survival of 100%.

The anti-viral effects (i.e. lower virus survival) of CPP and CPP3 are shown in tables 1-3.

TABLE 1

| | | % survival of A/Sydney/97 (H3N2) | | |
|---|---|---|---|---|
| | | | 1 hour pre- | 1 hour post- |
| | concentration | pre-incubation | exposure | infection |
| CPP | 10 μg/ml | 72 | 94 | 64 |
| CPP | 1000 μg/ml | 28 | 84 | 80 |
| CPP3 | 10 μg/ml | 62 | n.d. | n.d. |
| CPP3 | 1000 μg/ml | 66 | n.d. | n.d. |

(n.d. = not determined)

TABLE 2

| | | % survival A/Beijing/262/95 |
|---|---|---|
| | | (H1N1) |
| | | 1 hour post |
| CPP | 10 μg/ml | 70 |

TABLE 3

| | | % survival B/Harbin/07/94 |
|---|---|---|
| | | 1 hour post |
| CPP | 10 μg/ml | 90 |
| CPP | 1000 μg/ml | 74 |

### Example III In Vitro Data CPP Studies

### NK Cells Activity Test

NK-cells activity assay was performed on NK-92 cells as effector cells, and K562 cells (ATCC) as target cells. The specific lysis of target cells was determined by the lactate dehydrogenase (LDH) release assay using a cytotoxicity detection kit (Roche). Microtiter plates containing 200 μL of 5×10⁴ NK cells and 1×10⁴ K562 cells were incubated for 4 h at 37° C. in 5% CO₂ atmosphere. Spontaneous release of LDH was determined by incubation of cells into the medium alone. The effect of BSA, CPP and CPP3 on NK cells cytotoxicity activity was determined by adding the products in the medium at different concentrations (1, 10, 25 or 100 μg/mL). Maximum LDH release was obtained by incubating K cells (1×10⁴) into the medium containing 1% Triton X-100. Each product was evaluated in triplicate. Data were expressed as the percentage of cytotoxicity modulation:

$$( \frac{100 \times [ \begin{matrix}( \begin{matrix}N K : K + product - \\ N K + product\end{matrix} ) - \\ K + product\end{matrix} ]}{[ \begin{matrix}K maximum - \\ K + product\end{matrix} ]} ) - ( \frac{100 \times [ \begin{matrix}( \begin{matrix}N K : K medium - \\ N K medium\end{matrix} ) \\ - K medium\end{matrix} ]}{[ \begin{matrix}K maximum - \\ K medium\end{matrix} ] .} )$$

Results: BSA (control) did not alter cytotoxicity, whereas CPP and CPP3 enhanced NK cell cytotoxicity (table 4).

TABLE 4

| | Percentage cytotoxicity modulation at different concentrations | | | | | |
|---|---|---|---|---|---|---|
| | 1 | 10 | 25 | 100 | 500 | 1000 |
| | μg/ml | μg/ml | μg/ml | μg/ml | μg/ml | μg/ml |
| CPP | 16 | 10 | 9 | 1 | 8 | 9 |
| CPP3 | 17 | 26 | 24 | 22 | 21 | 20 |
| BSA | | −1 | | −1 | | |

### Example IV

### Animal Study 1

The challenge test consisted of the following steps
a) 3 groups of BALB/c mice, randomized based on weight (n=8 group)
b) Oral administration of a single daily dose of ingredient for 5 days pre-infection
c) Infection (administration of the influenza virus, 10³ PFU)
d) Oral administration of a single daily dose of ingredient for 5 days post-infection
e) Observations of the changes post-infection (symptoms & weight changes) up to 14 days post-infection

Mice were tube-fed with a daily dose of PBS (control, 0.5 mL), CPP3 (0.05 mg/g body weight in 0.3 mL PBS) or Echinacea (Echilin, Factors R&D Technology, 0.05 mg/g body weight in 0.3 mL PBS) during five days. Subsequently the mice were infected intranasally under light isofluran anaesthesia with influenza virus strain A/WSN/33 (10³ PFU, 40 μL PBS) followed by an additional 5 days of tube-feeding with the test products. Mice were sacrificed when they had lost 20% of their weight.

### Results:

Fourteen days after infection it was shown that one mouse (1/8) died in CPP3 supplemented group, and 2 mice died (2/8) in Echinacea group and three mice died (3/8) in the PBS group,
Furthermore a comparison of weight changes for each group was done and showed that the mice treated with CPP3 had a significantly lower body weight reduction up to day 9 post infection as compared to the Echinacea fed and control animals, indicating a better health for CPP3 treated animals.

### Example V

### Animal Study 2

The challenge test consisted of the following steps
a) 2 groups of BALB/c mice, randomized based on weight (n=12 group)
b) Oral administration of a single daily dose of ingredient for 5 days pre-infection
c) Infection (administration of the influenza virus, 10⁴ PFU) at day 0 (D0)
d) Oral administration of a single daily dose of ingredient for 5 days post-infection
e) Observations of the changes post-infection (symptoms & weight changes) up to 14 days post-infection

Mice were tube-fed with a daily dose of CPP3 (0.05 mg/g body weight in 0.3 mL PBS) or Echinacea (Echilin, Factors R&D Technology, 0.05 mg/g body weight in 0.3 mL PBS) during five days. Subsequently the mice were infected intranasally under light isofluran anaesthesia with influenza virus strain A/WSN/33 (10⁴ PFU, 40 μL PBS) followed by an additional 5 days of tube-feeding with the test products. Mice were sacrificed when they had lost 20% of their weight.

### Results:

No mice from Echinacea survived beyond day 6 of the experiment, whereas CPP3 group showed survival at day 6 and CPP3 group showed survival throughout experiment (table 5).

TABLE 5
Survival rate (%) of the experimental groups as a function of time
(days) during the challenge test until day 6 post infection

days post infection

| Treatment | 3 | 4 | 5 | 6 |
|---|---|---|---|---|
| CPP3 | 100 | 100 | 60 | 40 |
| Echinacea | 100 | 83 | 25 | 0 |

Table 6 summarizes weight data (at days 0 and 4) and shows the mean day of death for both groups. Both groups had a comparable average weight at day 0. The CPP3 mice induced no significant differences (considering the SD values) in terms of MDD or weight loss at day 4, in comparison with the Echinacea treated mice. Nevertheless, CPP3 had less weight loss and a higher mean day of death compared to Echinacea fed mice.

TABLE 6
Average weight, weight loss at D4 (%) and mean
day of death (MDD) for each experimental group

| | Weight at Day 0 | % of weight loss at | MDD |
|---|---|---|---|
| Groups | (±SD) | day 4 (±SD) | (±SD) |
| CPP3 | 17.5 ± 0.6 | 20.3 ± 7.7 | 6.8 ± 2.7 |
| Echinacea | 17.7 ± 1.0 | 23.5 ± 3.1 | 5.0 ± 0.6 |

### Example VI

### Animal Study 3

The challenge test consisted of the following steps
a) 2 groups of BALB/c mice, randomized based on weight (n=15 group)
b) Oral administration of a single daily dose of ingredient for 5 days pre-infection
c) Infection (administration of the influenza virus, 3*10³ PFU) at day 0 (D0)
d) Oral administration of a single daily dose of ingredient for 5 days post-infection
e) Observations of the changes post-infection (symptoms & weight changes) up to 14 days post-infection

Mice were tube-fed with a daily dose of CPP3 (0.05 mg/g body weight in 0.3 mL PBS) or Echinacea (Echilin, Factors R&D Technology, 0.05 mg/g body weight in 0.3 mL PBS) during five days. Subsequently the mice were infected intranasally under light isofluran anaesthesia with influenza virus strain A/WSN/33 (3*10³ PFU, 40 μL PBS) followed by an additional 5 days of tube-feeding with the test products. Mice were sacrificed when they had lost 20% of their weight.

### Results:

The results are summarized in table 7.

TABLE 7
Average weight at D0, weight loss D6 (%) and mean
day of death (MDD) for each experimental group

| | | weight at D0 | % of weight loss | |
|---|---|---|---|---|
| Groups | % mortality | (±SD) | at D6 post-inf | MDD |
| CPP3 | 73.3 | 21.1 ± 1.4 | −13.6 ± 11.2 | 10.9 ± 3.9 |
| EC3 | 93.3 | 21.2 ± 1.2 | −18.9 ± 8.6 | 6.8 ± 2.1 |

Table 7 shows that CPP3, in comparison with Echinacea, has higher survival rates and shorter mean day of death values. At day 6 after infection the CPP3 supplemented mice have lower body weight losses as compared to Echinacea fed animals.

1-12. (canceled)

13. A method for the prevention or treatment of virus infections comprising administering to a human or animal susceptible to or suffering from a virus infection an effective amount of one or more non-human casein peptides.

14. A nutritional or pharmaceutical composition containing casein peptide containing from 2 to 50 amino acid residues, together with a non-casein protein and/or a carbohydrate, the weight ratio between the casein peptide and the carbohydrate and/or protein being between 1:2 and 1:50.

15. A nutritional or pharmaceutical composition containing casein peptide containing from 2 to 50 amino acid residues, further containing at least one component selected from vitamin C, lactoferrin, transferrin, glycomacropeptide and echinacea.

16. The method according to claim 13, in which the casein peptide is a casein phosphopeptide (CPP).

17. The method according to claim 13, in which the casein peptide has an amino acid sequence of at least 2 up to about 50 amino acid residues.

18. The method according to claim 13, in which the casein peptide has from 3 up to about 25 amino acid residues.

19. The method according to claim 13, in which the casein peptide, on average, contains at least 1 phosphoserine residue per 20 amino acid residues.

20. The method according to claim 19, in which the casein peptide, on average, contains at least 1 phosphoserine residue per 10 amino acid residues.

21. The method according to claim 13, in which the casein peptide is part of a hydrolysate obtained by trypsin hydrolysis of casein.

22. The method according to claim 13, in which the casein peptide is used in an amount of 1 to 1000 mg per kg body weight per day.

23. The method according to claim 22, in which the casein peptide is used in an amount between 5 and 750 mg/kg per daily dosage unit.

24. The method according to claim 13, in which the composition further comprises lactoferrin or transferrin.

25. The method according to claim 13, wherein the virus is an influenza virus or a cold virus.

26. The method according to claim 13, wherein the casein is a Bovidae casein.

27. The method according to claim 24, wherein the casein is a bovine casein.
